(19) United States
(12) Patent Application Publication
(10) Pub. No.: US 2009/0017171 A1
(43) Pub. Date: Jan. 15, 2009

(54) PROOF AND PAN DOUGH TEMPLATE SYSTEM

(75) Inventors: Kevin Haas, St. Louis Park, MN (US); Joe SHEEHY, Chaska, MN (US)

Correspondence Address: GENERAL MILLS, INC., P.O. BOX 1113, MINNEAPOLIS, MN, 55440, US

(73) Assignee: General Mills Marketing, Inc.

(21) Appl. No.: 12/171,371

(22) Filed: Jul. 11, 2008

Related U.S. Application Data
(60) Provisional application No. 60/959,225, filed on Jul. 12, 2007.

(51) Int. Cl. A21D 10/02; A47J 37/00; G01N 33/02

(52) U.S. Cl. 426/113; 99/355; 426/233

(57) ABSTRACT

A proof and pan template system and related methods of use for consistently preparing proofed, baked products from unproofed dough units. The proof and pan template system can comprise at least one positioning template and a proofing template for verifying the arrangement and positioning of unproofed dough units on a baking pan prior to proofing. The positioning template can interface with the baking pan such that unproofed dough units can be placed and stretched appropriately prior to proofing. The measuring template can include a plurality of sides, wherein each side performs an independent measurement verifying the placement of the unproofed dough units. Following proofing, a proof height of the now proofed dough units can be confined with a proof area defined on the measuring template.

### CROSS-REFERENCE TO RELATED APPLICATIONS

This application claims the benefit of priority under 35 U.S.C. 119(e)(1) of a provisional patent application Ser. No. 60/959,225, filed Jul. 12, 2007, which is incorporated herein by reference in its entirety.

### TECHNICAL FIELD

The present disclosure is generally related to the preparation of proofed dough products. More specifically, the present disclosure is related to a proof and pan template system and related methods of use for preparing freshly baked, proofed dough products at a point of sale.

### BACKGROUND

In order to increase the enjoyment and loyalty of consumers, it has become increasingly frequent to prepare fresh baked goods on-site for use and consumption the same day. While such practices have been standard in bakeries for many years, the use of such techniques at sandwich and coffee shops has only become prevalent in recent years. As these style shops frequently have limited space for baking components and seldom if ever have experienced bakers on staff, the freshly baked goods are typically prepared simply by baking frozen dough units.

Unfortunately, the inexperience of the personnel can sometimes lead to unsatisfactory preparation resulting in baked goods that are not suitable for sale. Unsatisfactory preparation can include inadequate proofing of the dough units, overproofing of the dough units, inadequate spacing between dough units during baking and the like. Negative results associated with unsatisfactory preparation can include baked product sizes that are too large or too small as well as baked goods that have baked into adjacent units such that shapes and sized have become distorted. Any of these negative results can require disposal of the baked goods which consequently has a negative impact on efficiency and profitability.

In order to reduce waste and increase profitability, it would be advantageous to develop a tool that allows a relatively inexperienced employee to consistently and repeatedly prepare satisfactory baked goods.

### SUMMARY

The present disclosure is directed to a proof and pan template system and related methods of use for consistently preparing satisfactory proofed, baked products from unproofed or partially proofed (e.g. frozen, thawed, retarded, refrigerated) dough units. Generally, the proof and pan template system can comprise at least one placement template and a multi-functional proofing template useful for positioning unproofed dough units and testing proof height prior to baking. At least one proofing template can comprise a placement body divided into a plurality of body segments separated by a plurality of dividing elements. Generally, a first end spacing distance is defined between a first placement end and the nearest dividing element, a dough spacing distance is defined between adjacent dividing elements and a second end spacing distance is defined between a second placement end and the nearest dividing element. The proofing template can include a first side having two or more projecting legs defining a plurality of proof height measurement areas for testing and verifying that the dough unit is sufficiently proofed for baking. The proofing body can include a second side having a one or more spacer members for verifying spacing between unproofed dough units on a baking pan. The proofing body can further include a third side having a side projecting spacer for confirming spacing between sidemost unproofed dough units and a pan side. The template body can further include a fourth side having an end projecting spacer for confusing spacing between ends of the unproofed dough units and end surfaces of the baking pan. Utilizing the sides of the proof and pan template, a relatively inexperienced user can verify proper placement, spacing and proofing of dough units on the baking pan prior to baking, such that consistent and satisfactory baked products are produced.

In one aspect, the present disclosure is directed to a proof and pan template system for confirming the placement and spacing of unproofed dough units on a baking pan prior to proofing as well as confirming a proofed height of a proofed dough unit prior to baking. The proof and pan template system can comprise at least one placement template and a multi-purpose proofing template. In a representative embodiment, the proof and pan template system can comprise a pair of placement templates having a placement body divided into a plurality of body segments separated by a plurality of dividing elements. Generally, a first end spacing distance is defined between a first placement end and the nearest dividing element, a dough spacing distance is defined between adjacent dividing elements and a second end spacing distance is defined between a second placement end and the nearest dividing element. In a representative embodiment, the multi-purpose proofing template can include a first side having two or more projecting legs so as to define a plurality of proof height measurement areas that can be placed over a partially proofed or proofed dough for verifying that the previously unproofed dough unit is sufficiently proofed for baking. In another representative embodiment, the template body can include a second side having one or more spacer members for verifying unproofed dough unit spacing between adjacent unproofed dough units on a baking pan. In another representative embodiment, the template body can further include a pair of end projecting members, wherein each end projecting member is sized so as to confirm either the space between sidemost unproofed dough units and a pan side or for confirming spacing between ends of the unproofed dough units and end surfaces of the baking pan.

In another aspect, the present disclosure is related to a method for preparing consistently sized and shaped proofed dough products from a previously unproofed dough unit. Generally, the method can comprise providing at least one placement template on an end surface of a baking pan. A plurality of thawed and unproofed dough units can be positioned and stretched on the baking pan according to the at least one placement template. A multi-functional proofing template having a plurality of sides can be provided, wherein at least one of the sides is capable of providing a visually distinguishable measurement for verifying positioning of the unproofed dough units prior to proofing. In one embodiment, the method can comprise measuring a proof height of a proofed dough unit with the proofing template to verify that sufficient proofing is performed prior to baking. The method can further comprise verifying spacing between adjacent unproofed dough units with the proofing template prior to proofing. The method can further comprise verifying spacing around the sides and ends of the baking pan with the proofing template prior to proofing.

In another aspect, the present disclosure is related to a baking system for consistently preparing suitable baked dough products from unproofed dough units. In one representative embodiment, the baking system can comprise at least one positioning template, a multi-purpose proofing template and a baking pan. The positioning template can interface with the baking pan to provide an initial indication where thawed, unproofed dough units should be placed and to what degree the thawed dough units should be stretched prior to proofing. The multi-purpose proofing template which includes a plurality of sides having various visual measuring indicators can be used to verify that the thawed, unproofed dough units are properly positioned prior to proofing. In addition, the multi-purpose proofing template can provide a visual measurement of a proofed height of a partially proofed or proofed dough unit so as to confirm that sufficient proofing has occurred and that the dough unit is ready for placement on the baking pan in an oven. In some embodiments, the positioning templates can be integrally formed on the baking pan or on a liner sheet placed on the baking pan.

In another aspect, the present disclosure relates to a packaging assembly for shipping unproofed dough units in various states including frozen, refrigerated or ambient states. The packaging assembly can include a package containing a plurality of unproofed dough units. In some embodiments, a proof and pan template system can be shipped within the interior of the package. Alternatively, the proof and pan template system can be stamped or otherwise printed on the package such that a user can cut or punch the proof and pan template system from the package when the frozen dough units are to be prepared.

As used throughout the present disclosure, the term unproofed dough units refers to a dough composition including a leavening agent, wherein the dough composition is intended to undergo proofing prior to and/or during baking. Unproofed dough units can include previously frozen dough units that have been thawed in a retarder or under ambient conditions such that the proofing process has not been initiated or has only just commenced.

The above summary of the disclosure is not intended to describe each illustrated embodiment or every implementation of the present invention. The Figures and the detailed description that follow more particularly exemplify these embodiments.

### BRIEF DESCRIPTION OF DRAWINGS

The present disclosure and related invention may be more completely understood in consideration of the following detailed description of various embodiments of the invention in connection with the accompanying drawings, in which:

FIG. 1 is a top view of a proof and pan template system according to an embodiment of the present disclosure.

FIG. 1A is a top view of a multi-purpose proofing template according to an embodiment of the present disclosure.

FIG. 2 is a top view of a plurality of frozen dough units placed on a baking pan.

FIG. 3 is a top view of the baking pan of FIG. 2 including a pair of placement templates according to an embodiment of the present disclosure.

FIG. 4 is a top view of the baking pan of FIG. 3 including a plurality of thawed, unproofed dough units stretched as indicated by the placement templates of FIG. 4.

FIG. 5 is a side view of the baking pan of FIG. 4 including a plurality of proofed dough units having a proof height verified with a proofing template according to the present disclosure.

FIG. 6 is a side view of the baking pan of FIG. 5 including the plurality of unproofed dough units having an adjacent dough unit spacing verified with the proofing template.

FIG. 7 is a side view of the baking pan of FIG. 5 including a sidemost unproofed dough unit having a side spacing verified with the proofing template.

FIG. 8 is a side view of the baking pan of FIG. 5 including a sidemost unproofed dough unit having an end spacing verified with the proofing template.

FIG. 9 is a top view of the baking pan of FIG. 2 including a plurality of baked dough units.

FIG. 10 is a top view of a proof and pan template system having a pair of placement templates integral to a baking pan according to an embodiment of the present disclosure.

FIG. 11 is a perspective view of a packaging assembly including a proof and pan template system according to an embodiment of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

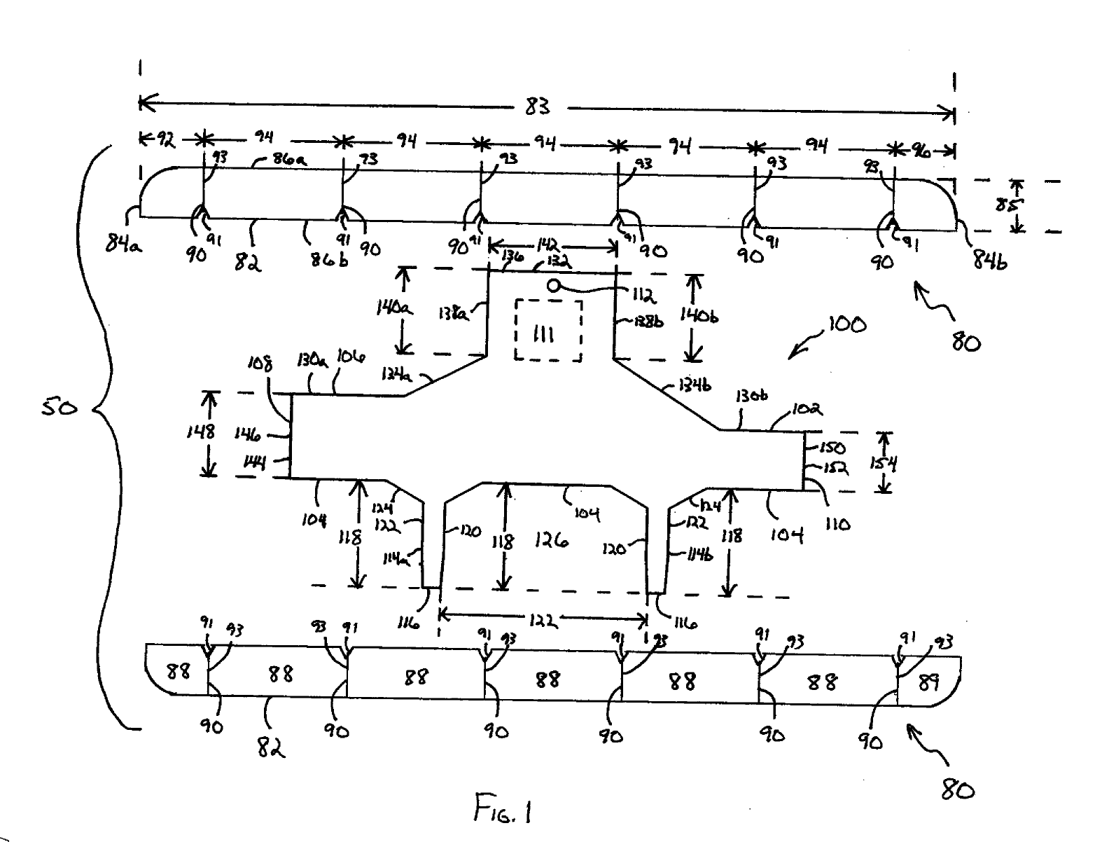

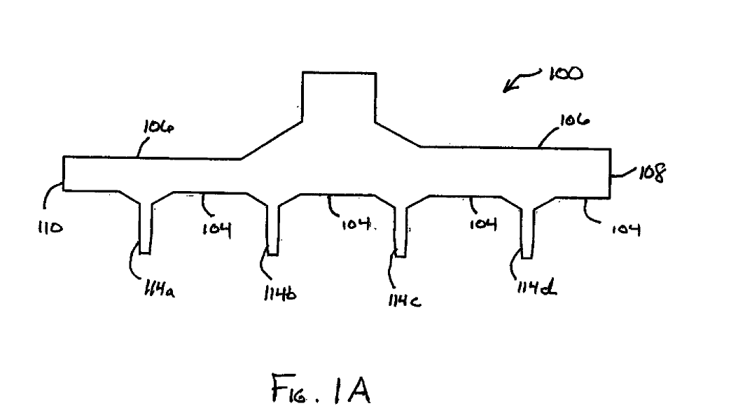

As illustrated in FIG. 1, a proof and pan template system 50 according to an embodiment of the present disclosure can comprise a pair of placement templates 80 and at least one multi-purpose proofing template 100. Placement template 80 and multi-purpose proofing template 100 can both be constructed from food-grade material such as, for example, a wood fiber product such as paper or cardboard, a food-grade polymer such as polycarbonate or a food-grade metal such as stainless steel or copper.

Referring to FIG. 1, placement template 80 generally comprises a placement body 82. Placement body 82 generally has a placement length 83 defined between a pair of placement ends 84a, 84b and a placement width 85 defined between a pair of placement sides 86a, 86b. Placement body 82 is divided into a plurality of body segments 88 defined by a plurality of visual elements 90. Visual elements 90 can include one or both of triangular notches 91 or dividing lines 93. Generally, a first end spacing distance 92 is defined between placement end 84a and the nearest dividing element 90, a dough spacing distance 94 is defined between adjacent dividing elements 90 and a second end spacing distance 96 is defined between placement end 84b and the nearest dividing element 90.

As illustrated in FIG. 1, multi-purpose proofing template 100 can comprise a proofing body 102 having a first side 104, a second side 106, a third side 108 and a fourth side 110. Depending upon the material used to construct proofing body 102, the measuring template can be fabricated using any of a variety of suitable methods including, for example, molding, punching, cutting and the like. Proofing body 102 can include a display area 111 for placing instructions or product information on the measuring template such that users can quickly identify the appropriate multi-purpose proofing template 100 for a particular dough product. Proofing body 102 generally has a body thickness that is preferably less than about 0.5 inches. Proofing body 102 can further include one or more apertures 112 extending through the body thickness and having a diameter sufficient to allow the multi-purpose proofing template 100 to be hung from a hook or similar device when not in use.

Referring again to FIG. 1, first side 104 generally comprises a first surface 113 having at least a pair of projecting arms 114a, 114b. Each projecting arm 114a, 114b, includes an arm end surface 116 such that a proof height 118 is defined between the arm end surface 116 and the first surface 113. Each projecting arm 114a, 114b further includes an inner arm surface 120 and an outer arm surface 122 wherein the inner arm surfaces 120 of adjacent projecting arms 114a, 114b is separated by proof width 122. Projecting arms 114a, 114b are generally configured for use with a specified dough unit such that proof height 118 and proof width 122 can be varied between product type. Projecting arms 114a, 114b can operably attach to the first surface 113 with an angled arm surface 124 such that the combination of inner arm surface 120, angled surfaces 124 and first surface 113 define a proof area 126 having the desired proof height 118 and proof width 122. Referring to FIG. 1A, an embodiment of multi-purpose proofing template 100 can comprise three or more projecting arms 114a, 114b, 114c, 114d so as to define additional proof areas 126.

As illustrated in FIG. 1, second side 106 is generally defined by a pair of non-planar second surfaces 130a, 130b, at least one projecting spacer 132 and a pair of angled spacer arms 134a, 134b. Projecting spacer 132 includes a projecting end surface 136 and a pair of projecting side surfaces 138a, 138b that connect the projecting end surface 136 with the corresponding non-planar second surfaces 134a, 134b. The projecting side surfaces 138a, 138b define spacer heights 140a, 140b between the projecting end surface 136 and the corresponding angled spacer arms 134a, 134b. Projecting spacer 132 has a spacer width 142 defined between the projecting side surfaces 138a, 138b.

With reference once again to FIG. 1, third side 108 generally defines a side spacer member 144 having a third surface 146. Side spacer member 144 generally has a side spacer width 148 defined between non-planar second surface 130a and first surface 113.

As illustrated in FIG. 1, fourth side 110 generally defines an end spacer member 150 having a fourth surface 152. End spacer member 150 generally has an end spacer width 154 defined between non-planar second surface 130b and first surface 113.

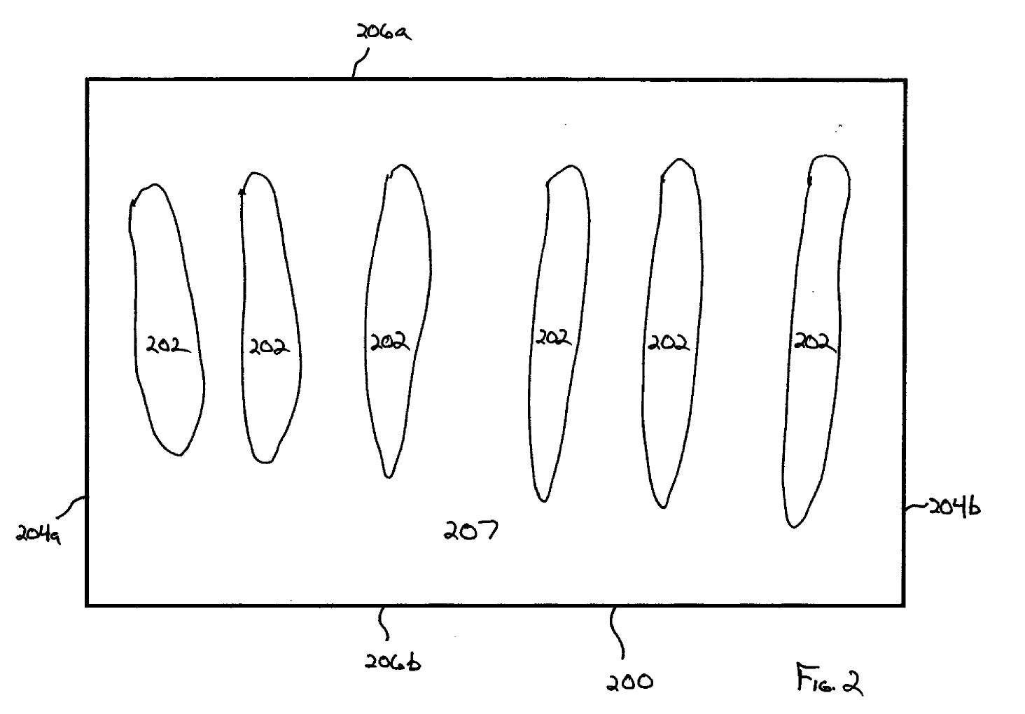

Use of proof and pan template system 50 is described generally with respect to a baking pan 200 and dough units 202 as illustrated in FIGS. 2-9. Generally, baking pan 200 can comprise a traditional cookie sheet or pan having a pair of pan side surfaces 204a, 204b, a pair of pan end surfaces 206a, 206b and a generally flat baking surface 207. Baking pan 200 generally resembles a rectangular or square configuration and the overall size is based upon the number and size of dough units 202 being prepared at any one time. In one representative embodiment, baking 200 can comprise a commercial baking pan having a width of 17.5″ and a length of 25.5″.

Dough units 202 generally comprise any of a variety of products that are prepared as fresh baked products from unproofed dough units. As presently contemplated, dough units 202 generally comprise products that experience an amount of proofing either during a distinct proofing step, during baking of the dough units or during a combination of these two steps. Dough units 202 of the present disclosure generally comprises a dough composition including leavening agents such as yeast, chemical or water/steam base leaveners including, for example, bread, rolls, breadsticks, pretzels, cinnamon rolls, croissants and the like. In one representative embodiment as illustrated in FIGS. 2 and 4-9, dough units 202 can comprise submarine sandwich rolls for use in preparing made-to-order sandwiches.

Generally, dough units 202 can be removed from a freezer and placed in a retarder or in ambient conditions to thaw. Generally, these conditions are sufficient to thaw the dough units 202 without activating the leavening agent that would otherwise commence the proofing process. Once thawed, the dough units 202 can be placed onto baking pan 200. Once placed on baking pan 200, the dough units 200 begin to warm resulting in the yeast being activated, resulting in the formation of carbon dioxide within dough units 202. The presence of carbon dioxide causes the dough units 202 to increase in size and volume, sometimes by two times their original size and volume. As the dough units 202 undergo this proofing process, growth in the size and volume of the dough units 202 can lead to physical interaction of adjacent dough units that can negatively impact shape and appearance of the dough units 202. In order to achieve satisfactory baked results for dough units 202, placement template 80 can be used to verify proper placement and positioning of the originally unproofed dough units 202 prior to activation of the proofing process. Multi-purpose proofing template 100 can be similarly used to verify positioning of the dough units 202 prior to proofing as well as measuring a proofed height of the dough units 202 to verify that sufficient proofing has occurred prior to placement of the baking pan 200 and dough units 202 within an oven. Use of placement template 80 and multi-purpose proofing template 100 provides a user the ability maximize the placement of dough units 202 on the baking pan 200 while preventing adjacent dough units 202 from proofing into one another.

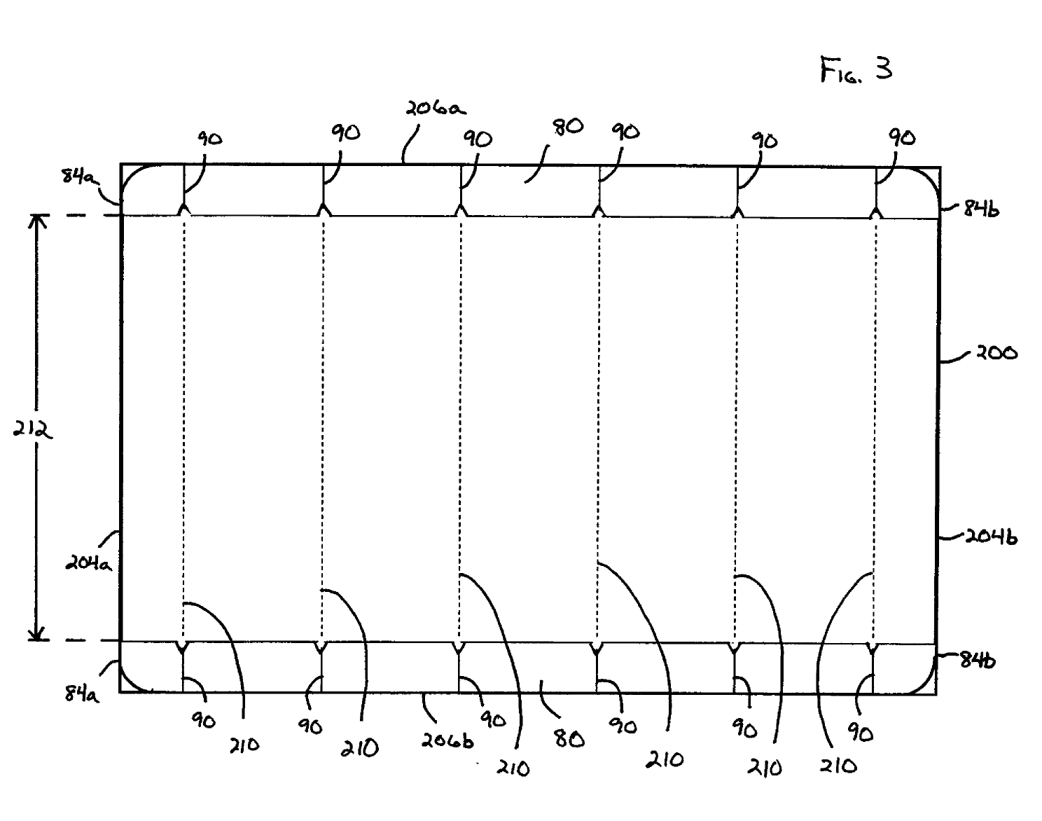

Generally, baking pan 200 is placed on a generally flat counter or table such that an operator can place dough units 202 on the baking pan 200 as illustrated in FIG. 2. The placement templates 80 are generally arranged along the pan end surfaces 206a, 206b such that the placement ends 84a, 84b are even with the pan side surfaces 204a, 204b while one of the placement sides 86a, 86b on each placement template 80 resides along one of pan end surfaces 206a, 206b as illustrated in FIG. 3. In this manner, placement templates 80 are arranged in a substantially parallel orientation with the dividing elements 90 aligned so as to define a plurality of placement lines 210 that are generally perpendicular to the pan end surfaces 206a, 206b. In addition, each placement line 210 has a placement length 212 that corresponds to a desired stretch length for the dough units 202 prior to baking.

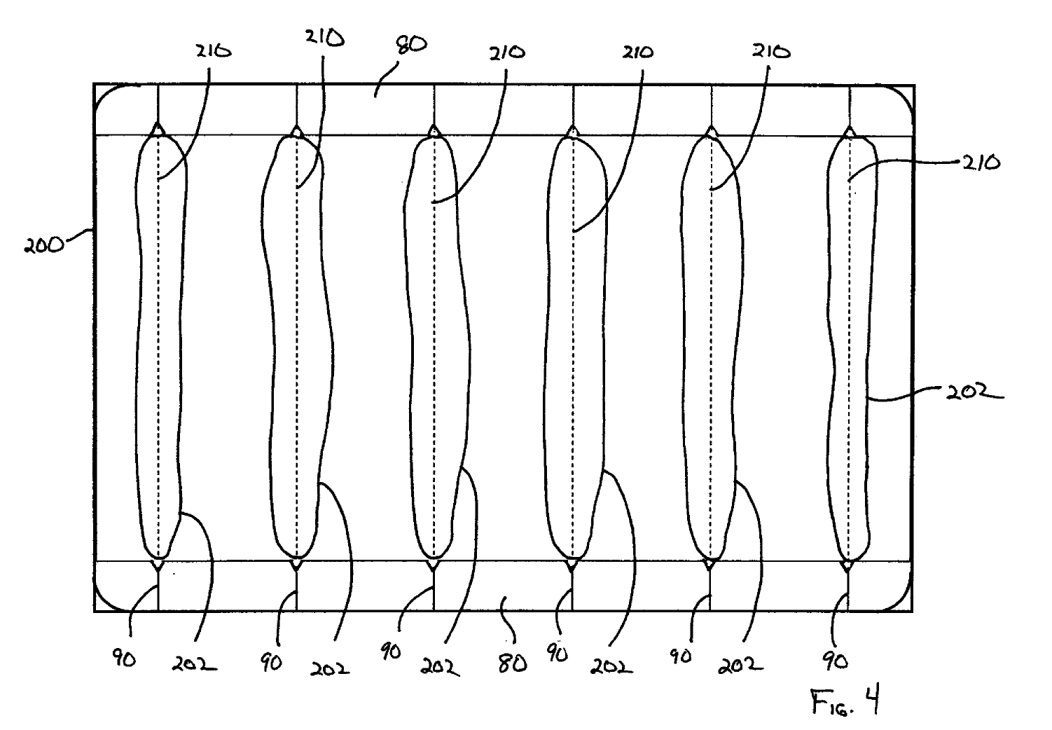

With the placement templates 80 arranged as described above and as illustrated in FIG. 3, an individual thawed, unproofed dough unit 202 is placed along the placement line 210, wherein the dough unit 202 can then be stretched to equal placement length 212 as illustrated in FIG. 4. With the dough units 202 spaced at either first end spacing distance 92, dough spacing distance 94 or second end spacing distance 96, each dough unit 202 has enough room to fully proof and expand such the overall size and appearance of baked dough units are equal and consistent. As will be understood by one of skill in the art, the various spacing distances and placement length can vary between dough products based upon the baked product being prepared such that each baked product requires its own specific set of placement templates 80. Once dough units 202 have been positioned and stretched along placement lines 210, the placement templates 80 can be removed from baking surface 207.

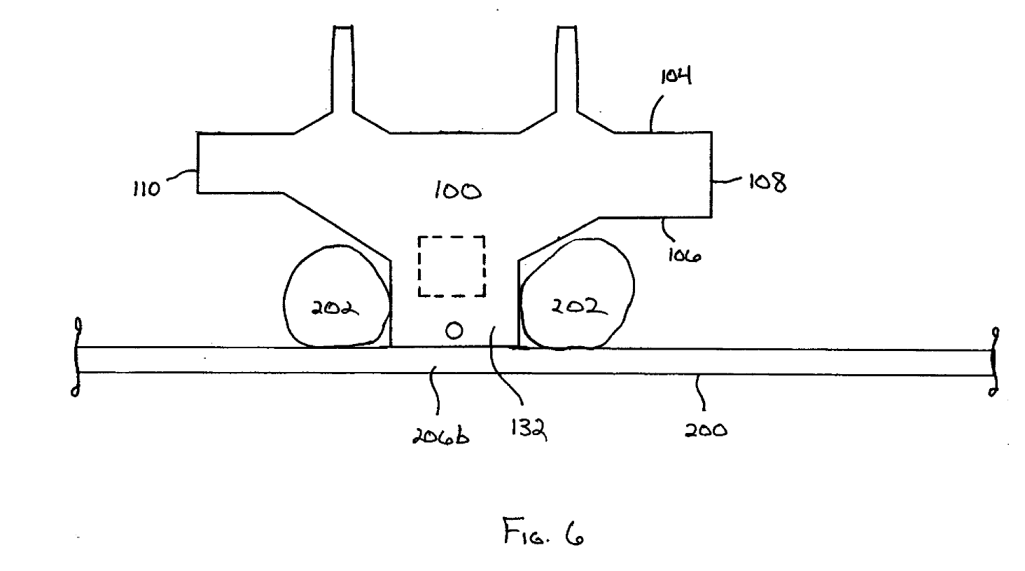

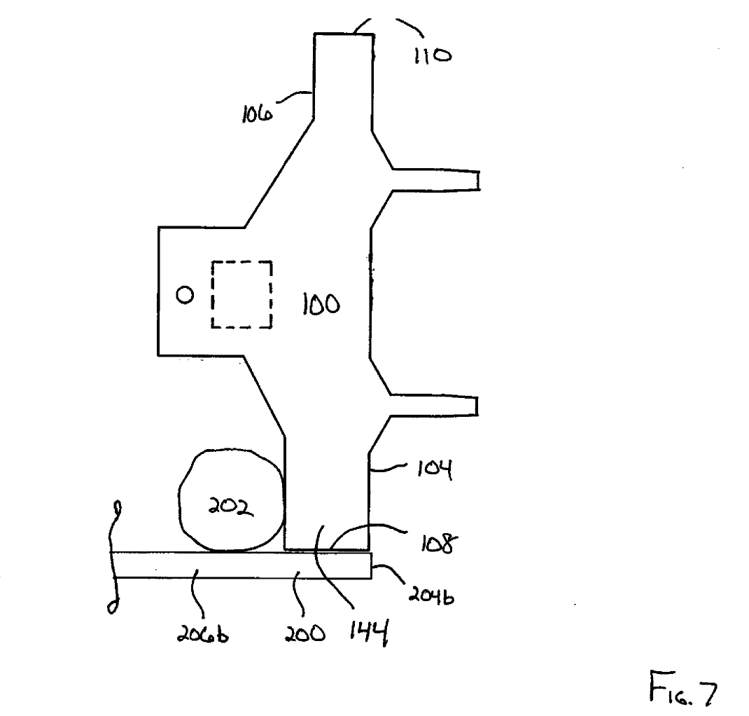

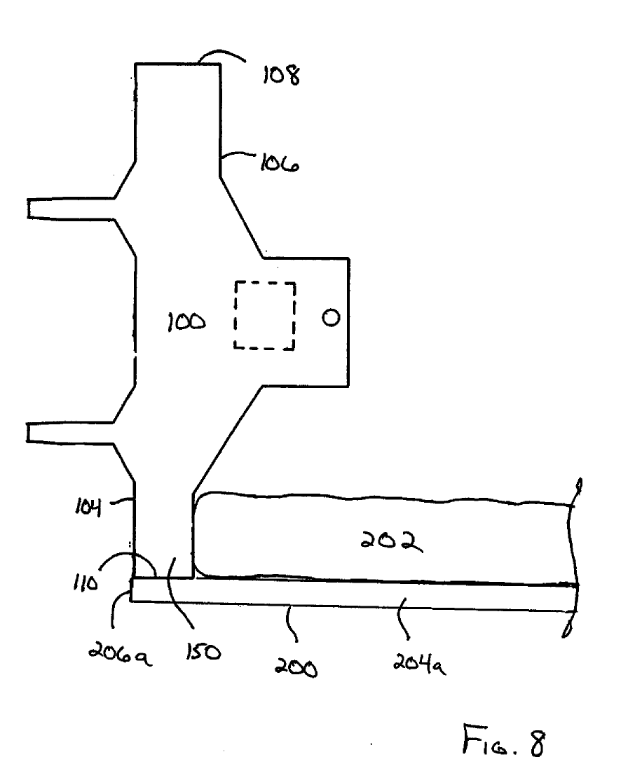

Referring to FIGS. 6, 7 and 8, multi-purpose proofing template 100 can be use to confirm the positioning and stretch length of the unproofed dough units 202 accomplished using placement templates 80. Referring to FIG. 6, multi-purpose proofing template 100 can be utilized to confirm spacing between adjacent unproofed dough units by placing the projecting spacer 132 between adjacent dough units 202. By verifying that adjacent unproofed dough units 202 are spaced apart by at least spacer width 142, the user can be assured that the adjacent dough units 202 do not expand and proof into each other as proofing occurs. Referring to FIG. 7, multi-purpose proofing template 100 can be oriented such that side spacer member 144 confirms a spacing between pan side surfaces 204a, 204b and the two unproofed dough units 202 lying generally parallel to the pan side surfaces 204a, 204b. By verifying that spacing between the pan side surfaces 204a, 204b and the two parallel unproofed dough units 202 exceeds at least side spacer width 148, the user can be assured that the two parallel dough units 202 will not expand off the flat baking surface 207 during the proofing process. Referring to FIG. 8, multi-purpose proofing template 100 can be oriented such that end spacer member 150 confirms a spacing between pan end surfaces 206a, 206b and the ends of unproofed dough units 202. By verifying that the spacing between the pan end surfaces 206a, 206b and the ends of the unproofed dough units 202 exceeds at least end spacer width 154, the user can be assured that the ends of the dough units 202 will not expand off the flat baking surface 207 as proofing and/or baking is completed as well as confirming the stretch length of the unproofed dough units 202.

Once the dough units 202 have been placed on baking pan 200 using placement templates 80 and in some embodiments, confirmed using multi-purpose proofing template 100, the baking pan can be proofed either at ambient conditions on the counter or in a controlled temperature/humidity environment as found in a proof box. In one representative embodiment, dough units 202 can be allowed to reach a temperature of about 55° F. to about 60° F., wherein the baking pan 200 and dough units 202 can be placed in the proof box. Depending upon the product type and temperature/humidity conditions, the dough units 202 can be proofed for a desired period of time that generally ranges from about 30 to about 90 minutes. As discussed previously, the size and volume of the dough units 202 increase as this proofing occurs. Based either upon a visual check of the dough units 202 or following a designated proofing time, the user can verify that sufficient proofing has occurred using multi-purpose proofing template 100.

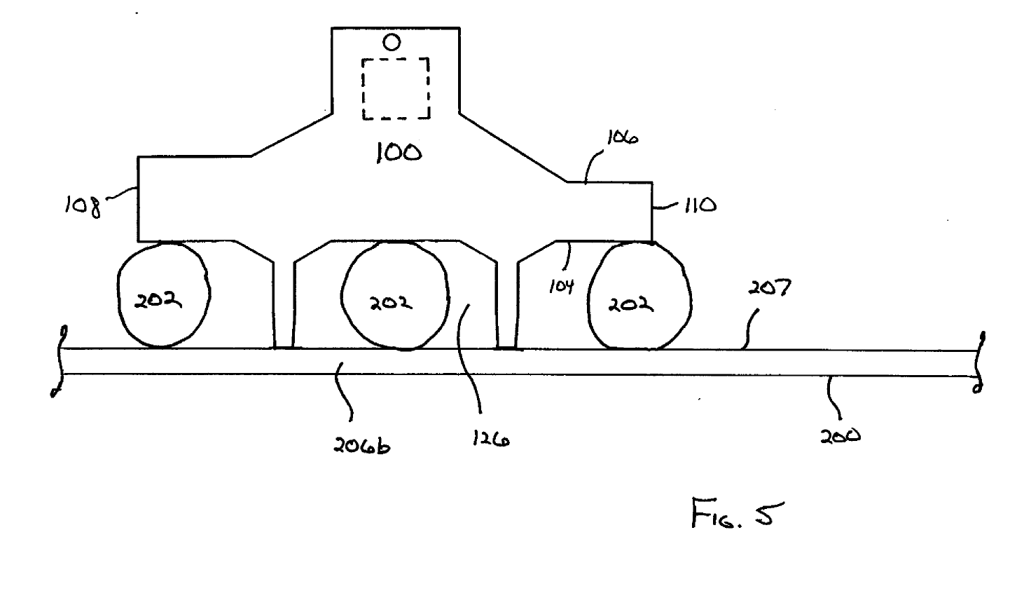

Once the dough units 202 have been stretched as illustrated in FIG. 4 and the user believes the dough units 202 have proofed for a sufficient time, the multi-purpose proofing template 100 is positioned as illustrated in FIG. 5 such that proof area 126 is arranged over one of the proofed dough units 202. When the proofed dough units 202 have reached proof height 118 as illustrated in FIG. 1, the user knows that the baking pan 200 and proofed dough units 202 are ready for baking. As illustrated in FIG. 5, multi-purpose proofing template 100 allows for verification of the proof height 118 of three adjacent proofed dough units 202. In this way, multi-purpose proofing template 100 need not be repositioned to place proof area 126 over each individual proofed dough unit 202. In this manner, the six dough units 202 illustrated in FIG. 4 can have their proof height 118 measured by positioning the multi-purpose proofing template 100 only twice. In the event that proofed dough units 202 have not achieved proof height 118, the proofing process can be extended.

Measurement of the proof height 118 is generally a pass/fail test with a pass condition being met by proofed dough units 202 achieving proof height 118. In some embodiments, proofed dough units 202 can achieve proof height 118 without being proofed for the full period of time suggested for the proofing process. In any event, once the proofed dough units 202 reach proof height 118, the proofed dough units 202 are ready for baking. In some embodiments, projecting arms 114a, 114b can be tapered proximate the arm end surface 116 to assist in preventing the projecting arms 114a, 114b from puncturing and disturbing the structure of the proofed dough units 202 during measurement of the proof height 118. In some alternative embodiments, projecting arms 114a, 114b can include a measuring element having English or metric measuring scales allowing a user to measure a height difference between proofed dough units 202 and first surface 113. The height difference can then be compared to a look up table where based upon the height difference and proofing conditions such as, temperature and humidity, the user can estimate the remaining proofing time until the proofed dough units 202 reach proof height 118.

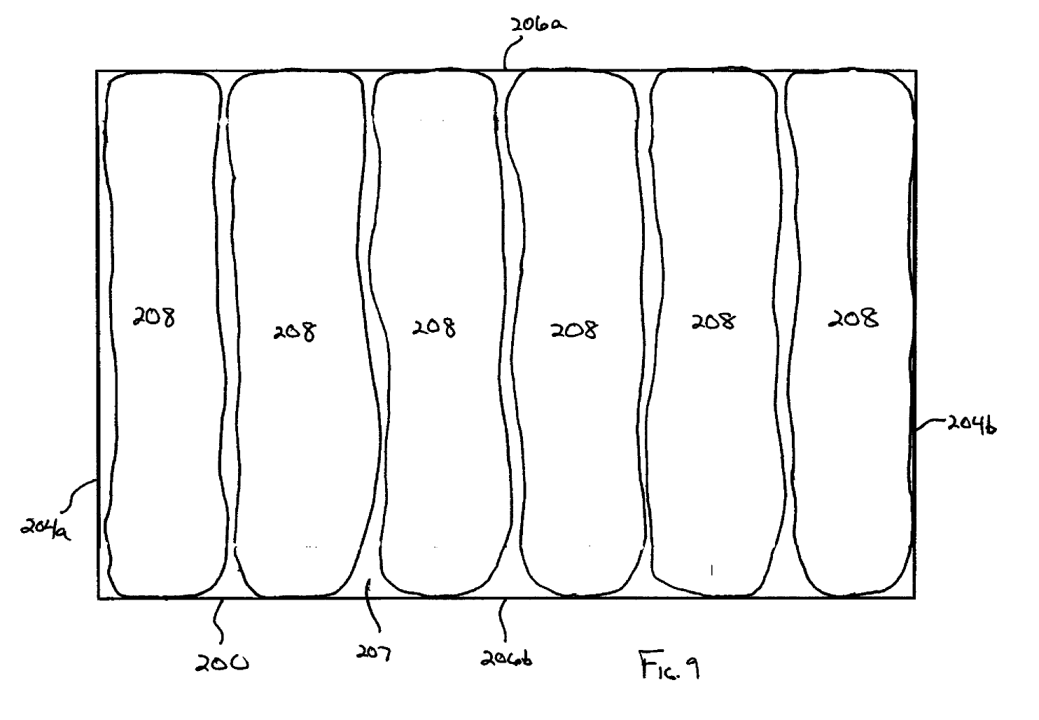

Following confirmation that the proofed dough units 202 have achieved proof height 118, the baking pan 200 including the dough units 202 can be placed in an oven for baking. Generally, the oven can comprise any of a variety of suitable ovens including toaster ovens, convection ovens and the like. Following baking, the baking pan 200 can be removed with baked dough units 208 as illustrated in FIG. 9. Due to the artisan nature of dough units 202, the baked dough units 208 will not be identical in appearance but will generally share the same overall length, width and volume.

Through the use of proof and pan template system 50, the user can verify both original positioning and stretch length on a baking pan 200 prior to proofing. In addition, the proof height 118 of proofed dough units 202 can be confirmed immediately prior to baking. In this manner, consistency in appearance and size can be maintained both within a single batch as well as between multiple batches of baked dough units.

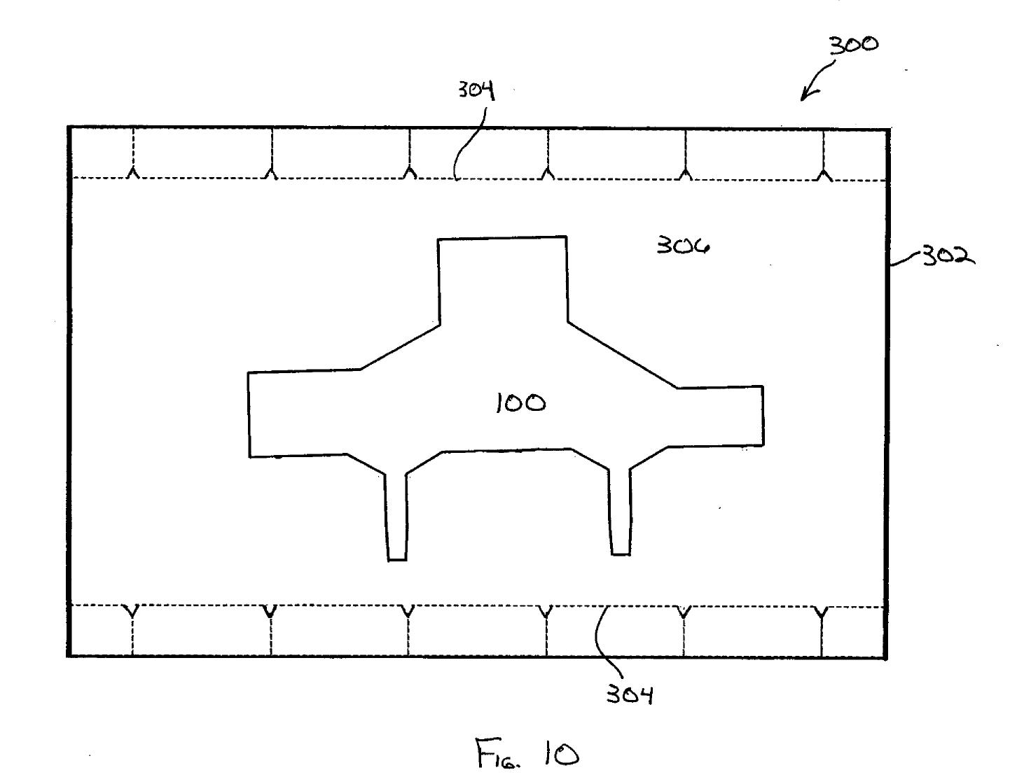

Referring now to FIG. 10, an alternative embodiment of a proof and pan template system 300 can comprise a baking pan 302 and at least one least one multi-purpose proofing template 100. Baking pan 302 can substantially resemble baking pan 200 in both materials of construction as well as size and shape. Baking pan 302 can further comprise an integral placement template 304 etched or otherwise formed and bonded to a baking surface 306. Integral placement template 304 can substantially resemble placement body 82 in both look and performance with the exception that integral placement template 304 is integral to baking pan 302. Similarly to placement body 82, baking pan 302 can include a pair of integral placement template bodies 304 located on opposed sides of the baking pan 302 so as to provide an alignment guide for placement of dough units 202 prior to proofing. Dough units 202 can be similarly positioned and proofed on baking pan 302 using integral placement template 304 and multi-purpose proofing template 100 as previously described with respect to proof and pan template system 50.

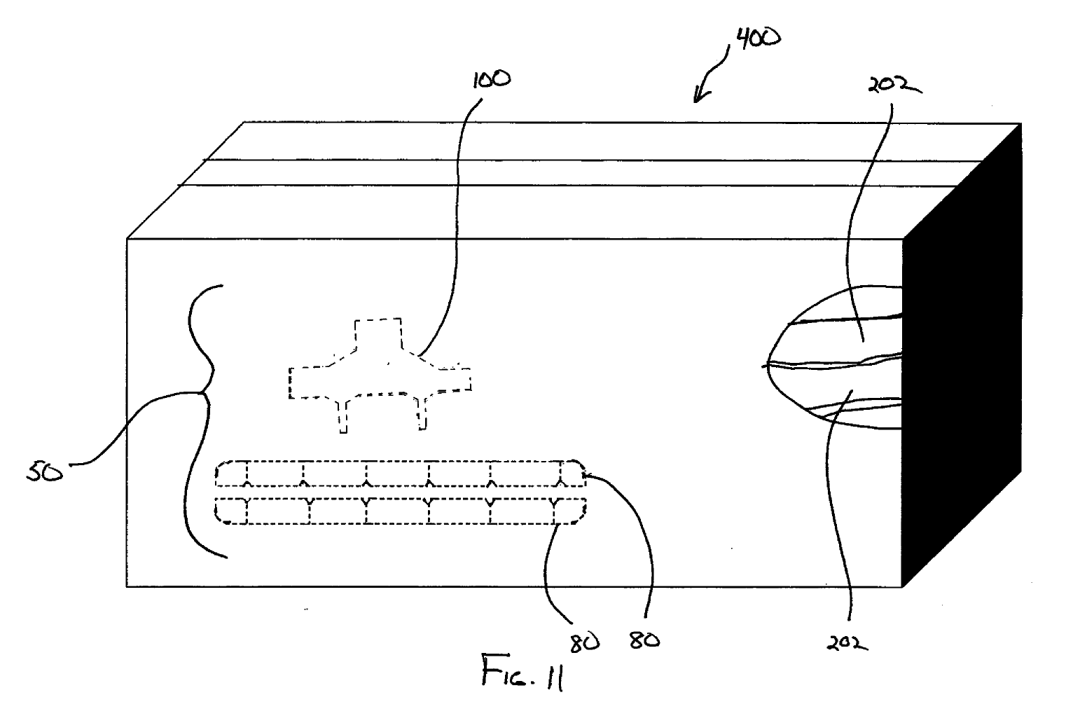

Proof and pan template system 50 can be provided as reusable components that can undergo typical washing and sanitization procedures. Alternatively, proof and pan template system 50 can be provided as essentially disposable components shipped with package 400 of frozen dough units 202 as illustrated in FIG. 11. In some embodiments, placement templates 80 and multi-purpose proofing template 100 can be fabricated prior to packaging and be included within package 400. For instance, placement template 80 and multi-purpose proofing template 100 can be dye cut or perforated in paperboard for removal at time of use. Alternatively, placement templates 80 and multi-purpose proofing template 100 can be essentially stamped, printed, dye cut, perforated or otherwise formed directly on package 400 for subsequent removal by a user. For instance, when package 400 comprises cardboard, placement templates 80 and multi-purpose proofing template 100 can be cut or punched from package 400 when package 400 is opened and the frozen dough units 202 are to be prepared.

While the invention has been described in connection with what is presently considered to be the most practical and preferred embodiment, it will be apparent to those of ordinary skill in the art that the invention is not to be limited to the disclosed embodiment, that many modifications and equivalent arrangements may be made thereof within the scope of the invention, which scope is to be accorded the broadest interpretation of the appended claims so as to encompass all equivalent structures and products.

What is claimed is:

1. A proof and pan template system for positioning dough intermediates on a baking surface comprising:
at least one dough placement template operably divided into a plurality of body segments and a pair of end segments, wherein adjacent body segments and end segments are distinguished by a visual element; and
at least one dough proofing template having a first side including a pair of projecting arms terminating at an arm end surface, and wherein a proof height is defined between the arm end surface and the first side.

2. The proof and pan template system of embodiment 1, comprising a pair of dough placement templates, each template being placed on an opposing side of a baking pan such that corresponding visual elements are aligned for providing a visual indication for positioning of dough units.

3. The proof and pan template system of embodiment 1, wherein the at least one dough placement template is formed integrally on a baking surface of a baking pan.

4. The proof and pan template system of embodiment 1, wherein the at least one dough proofing template includes a second side having a projecting spacer for measuring spacing between adjacent dough units.

5. The proof and pan template system of embodiment 4, wherein the at least one dough proofing template includes a third side having a side spacer member for measuring spacing between sidemost dough units and sides of a baking surface.

6. The proof and pan template system of embodiment 5, wherein the at least one dough proofing template includes a fourth side having an end spacer member for measuring spacing between ends of the dough units and end of the baking surface.

7. The proof and pan template system of embodiment 1, wherein the first side includes at least three projecting arms.

8. The proof and pan template system of embodiment 1, wherein the at least one dough placement template and the at least one dough proofing template are formed from a food-grade material selected from the group consisting of: a food-grade wood fiber product, a food-grade polymer and a food-grade metal.

9. The proof and pan template system of embodiment 1, wherein the visual element is selected from the group consisting of: a notched portion, a dividing line and combinations thereof.

10. A method for improving consistency of baked goods formed from proofed dough units comprising:
providing at least two unproofed dough units;
placing the at least two thawed dough units on a baking pan such that the unproofed dough units are spaced apart and aligned with a visual element on a dough placement template;
verifying a spaced apart distance between the at least two unproofed dough units with a projecting spacer on a proofing template;
proofing the at least two unproofed dough units to form at least two proofed dough units;
confirming a proof height of the at least two proofed dough units with a proof area defined on the proofing template; and
baking the at least two proofed dough units to form at least two baked goods.

11. The method of embodiment 10, further comprising:
stretching the at least two thawed dough units to a stretched length measured between opposed visual elements on a pair of dough placement templates.

12. The method of embodiment 10, further comprising:
forming the baking pan such that the dough placement template is integral to the baking pan.

13. The method of embodiment 10, further comprising:
providing the dough placement template and the dough proofing template within a container of the unproofed dough units.

14. The method of embodiment 10, further comprising:
providing an outline of the dough placement template and the dough proofing template on a container of the unproofed dough units; and
removing the dough placement template and the dough proofing template from the container.

15. The method of embodiment 10, further comprising:
verifying an edge spacing between the at least two unproofed dough units with the proofing template.

16. The method of embodiment 10, wherein confirming the proof height of the at least two proofed dough units with the proofing template comprises simultaneously confirming the proof height of at least three proofed dough units.

17. A packaging assembly comprising:
a package enclosing a plurality of unproofed dough units; and
a proof and pan template system including at least one positioning template and a proofing template.

18. The packaging assembly of embodiment 17, wherein the at least one positioning template and the proofing template are enclosed within the package.

19. The packaging assembly of embodiment claim 17, wherein the at least one positioning template and the proofing template are removably formed on a surface of the package.
